(19) United States
(12) Patent Application Publication
(10) Pub. No.: US 2024/0408333 A1
(43) Pub. Date: Dec. 12, 2024

(54) VENTILATOR, PROCESS FOR CONTROLLING A VENTILATOR, SYSTEM, COMPUTER PROGRAM PRODUCT AND COMPUTER-READABLE MEDIUM

(71) Applicant: Drägerwerk AG & Co. KGaA, Lübeck (DE)

(72) Inventor: Bastian SCHULDT, Lübeck (DE)

(21) Appl. No.: 18/735,438

(22) Filed: Jun. 6, 2024

(30) Foreign Application Priority Data
Jun. 9, 2023 (DE) 10 2023 115 140.1

(51) Int. Cl. A61M 16/00

(52) U.S. Cl. CPC A61M 16/022; A61M 2016/0027; A61M 2016/0033; A61M 2205/3355

(57) ABSTRACT

A ventilator, a process for controlling a ventilator, a system, and a computer program and computer readable medium is provided for ventilating a patient through an inspiratory line (10). The ventilator includes a breathing gas source (70) for supplying breathing gas, a controller (60) which acts as an actuator on the breathing gas source (70), a first sensor (20) configured to provide a pressure signal which indicates a pressure (p) in the inspiratory line (10), and a second sensor (30) configured to provide a volume flow signal which indicates a volume flow (V) in the inspiratory line (10). An expected pressure (p2) and/or an expected volume flow (V2) that is likely to occur are determined as to a maximum allowable pressure and/or volume flow to provide a determination result (E). A controller (60) regulates the pressure (p) and/or the volume flow (V) based on the determination result (E).

### CROSS REFERENCE TO RELATED APPLICATIONS

This application claims the benefit of priority under 35 U.S.C. § 119 of German Application 10 2023 115 140.1, filed Jun. 9, 2023, the entire contents of which are incorporated herein by reference.

### TECHNICAL FIELD

The present invention relates to a ventilator, a process for controlling a ventilator, a system, a computer program product and a computer-readable medium.

### BACKGROUND

It is a general requirement for ventilators that the patient should be ventilated gently, i.e. without damaging the patient's lungs in particular.

DE 10 2020 121 871 A1 discloses a ventilator for ventilating a patient by means of high-flow oxygen therapy via a tube system. This ventilator has at least one controllable inspiratory or expiratory valve which can be controlled by a control unit and wherein a ventilation pressure is regulated via the at least one inspiratory or expiratory valve in such a way that a predetermined maximum pressure is not exceeded in a predetermined area of the tubing system. If the predetermined maximum pressure is reached, the inspiration or expiration valve is consequently opened in order to prevent a further increase in pressure. In this state, the breathing gas can escape into the environment via the at least one inspiration or expiration valve.

EP 4 151 261 A1 discloses a ventilator comprising a nasal interface and a gas line for supplying a gas flow to an outlet of the nasal interface and a flow controller for selectively controlling the flow of gas into the nasal interface, the flow controller being configured to operate when a pressure in the system is above a predetermined value to restrict or prevent the flow of gas into the nasal interface from the gas line.

### SUMMARY

It is an object of the invention to provide an improved ventilator and a corresponding process for controlling a ventilator, a system, a computer program product and computer-readable medium.

This task is solved by a ventilator, a system, and a computer program and computer-readable medium according to the invention.

This disclosure, including the description, the drawings and the claims, presents advantageous embodiments of the invention.

According to the invention, a ventilator is provided for ventilating a patient through an inspiratory line. The ventilator comprises a first sensor which is adapted to provide a pressure signal indicating a pressure in the inspiratory line. The ventilator further comprises a second sensor which is configured to provide a volume flow signal indicating a volume flow (volume flow rate) in the inspiratory line. The ventilator is configured to determine a flow resistance of the inspiratory line based on the pressure signal and on the volume flow signal, to receive first information indicating a predetermined target volume flow and/or to receive second information indicating a predetermined maximum allowable inspiratory pressure, to determine an expected pressure based on the flow resistance and on the first and/or second information and to determine, whether the pressure expected to be established is higher than the maximum allowable inspiratory pressure and/or to determine a volume flow expected to be established based on the flow resistance and on the first and/or second information, and to determine whether the volume flow expected to be established is higher than a maximum allowable volume flow corresponding to the maximum allowable inspiratory pressure, and to provide a determination result corresponding to the determination. The ventilator also has a breathing gas source for supplying breathing gas and a controller (regulator) which acts as an actuator on the breathing gas source. The controller is configured to regulate the pressure and/or the volume flow based on the determination result in such a way that the maximum allowable inspiratory pressure and/or the maximum allowable volume flow is not exceeded.

In this way, it can be reliably ensured that a maximum allowable pressure and/or volume flow is not exceeded. Compared to known ventilators, in which a countermeasure or a reaction to the exceeding of a maximum allowable volume flow only takes place after it has been exceeded, the patient safety of the ventilator can thus be further improved.

Furthermore, by regulating the pressure and/or volume flow in such a way that the maximum allowable volume flow is already prevented from being exceeded on the device side, there is also no need to allow the respiratory gas conducted to the patient through the inspiratory line to escape into the environment via a valve. Compared to known devices that cannot do this, the gas consumption of the ventilator can therefore be advantageously reduced.

An expected pressure or volume flow is understood to be the pressure or volume flow that would occur as a result of the application of the predetermined target volume flow in the inspiratory line. In other words, a predictive determination of a corresponding pressure or volume flow is made and information is obtained as to whether this pressure or volume flow can be achieved within the predetermined limits.

The determination result can be processed in various ways. For example, the determination result can be output via an output unit so that it can be perceived by a user. The output unit can, for example, be a display unit of the ventilator or a device connected to the ventilator. The output unit can additionally or alternatively be configured to provide an acoustically perceptible output.

The user can thus be prompted to take appropriate measures, such as reducing the predetermined set flow rate and/or increasing the maximum allowable inspiratory pressure.

The determination result is also processed as an input variable for controlling the ventilator, for example by a control unit (that comprises one or more processor and a memory unit) of the ventilator.

The inspiratory line of the ventilator can be configured as an integral part of the ventilator or can be configured separately from the ventilator and can be connected to the ventilator. In a variant of the invention, the inspiratory line can be a line of a tubing system which is configured, for example, as a single-tube (single-hose) system, i.e. with only one inspiratory line, or as a two-tube (two-hose) system, i.e. with both an expiratory line and an inspiratory line. Further lines as part of the tube system are possible.

The first sensor and/or the second sensor can each be configured as integral components of the ventilator and/or as integral components of the inspiratory line and/or the tubing system and/or a patient interface. All that is required is that both the first sensor and the second sensor are connected to the ventilator via data technology in order to provide the ventilator with the respective pressure signal or volume flow signal.

In the context of the invention, the determination of a number of output variables based on a number of input variables is understood to mean that the number of input variables is converted into the number of output variables by an essentially arbitrary, predetermined data-processing process. When carrying out the data-processing process, it is possible to process additional information, such as additional input variables and/or predetermined parameters, in addition to the number of input variables. However, it is also possible to consider only the number of input variables.

The controller can be implemented using any control structure, for example by forming one or more linearly connected individual controllers or by forming cascaded connected individual controllers. The controller can be configured as a single-variable controller or as a multi-variable controller.

The controller can be implemented as a software module or as a plurality of software modules in the ventilator, for example on the control unit of the ventilator. Additionally, or alternatively, the controller can be implemented as an (analog and/or digital) hardware module in the ventilator.

In the context of the invention, a breathing gas source is understood to be a component or assembly of the ventilator which provides a gas or gas mixture intended for inspiration, namely the breathing gas, with suitable parameters on the device side, i.e. which specifically influences a volume flow and/or pressure of the gas conducted through the breathing gas source. In this respect, examples of breathing gas sources according to the invention are blowers and/or valves, in particular proportional valves. In this respect, an inspiratory valve and/or an expiratory valve is not a breathing gas source within the meaning of the invention. If an inspiratory valve is provided in or on the ventilator—which is possible—the breathing gas source is provided upstream of the inspiratory valve and is different from the inspiratory valve.

The flow resistance can be determined based on the pressure signal and the volume flow signal, for example, using a model of the flow resistance that has at least the pressure signal and the volume flow signal as input variables. Various degrees of abstraction of such a model are possible. A simple model of the flow resistance results, for example, from the following formula:

$$R = \frac{p}{{V}^{2}} ,$$

where R is the flow resistance, p is the pressure in the inspiratory line and V is the volume flow in the inspiratory line.

Another example of determining the flow resistance based on the pressure signal and the volume flow signal is the use of a lookup table, in which corresponding pre-calculated flow resistances are stored depending on the pressure signal and/or the pressure in the inspiratory line and depending on the volume flow signal and/or the volume flow in the inspiratory line. Compared to determining the flow resistance using a model, complex calculations can therefore be omitted and replaced by a less computationally intensive value search.

In the context of the invention, predetermined information may, for example, be predetermined by a therapy to be carried out by or by means of the ventilator and/or according to physiological parameters of a respective patient and may be selected and provided by the ventilator and/or by an operator of the ventilator for the respective therapy. Furthermore, for example, predetermined information may be information entered and/or selected by the operator on the ventilator or on a device connected to the ventilator by data technology, thus forming the predetermined information.

For example, for a high-flow oxygen therapy that can be carried out by means of the ventilator according to the invention, a target oxygen concentration of the respiratory gas conducted to the patient through the inspiratory line, a desired volume flow as a predetermined target volume flow and a predetermined maximum allowable inspiratory pressure are usually specified and taken into account by the ventilator.

Preferably, the ventilator is configured: to determine a first difference between the predetermined target volume flow and the maximum allowable volume flow, and to provide the first difference as a first input variable, in particular as a disturbance variable, for the controller.

In other words, the determination result preferably includes the first difference and can thus be taken into account by the controller in order to effectively prevent the maximum allowable volume flow from being exceeded.

The controller or the control unit of the ventilator is particularly preferably configured to determine the first difference.

In addition, or as an alternative to the preferred feature described above, it is preferred that the ventilator is configured: to determine a second difference between the maximum allowable inspiratory pressure and the pressure in the inspiratory line, and to provide the second difference as a second input variable, in particular as a factor of a proportional component of the controller, for the controller.

In other words, the determination result preferably (additionally or alternatively) comprises the second difference and can thus be taken into account by the controller in order to effectively prevent the maximum allowable volume flow from being exceeded.

If the second difference is provided as a factor of a proportional component of the controller, a continuously degressive approximation of the volume flow and/or the pressure to the respective maximum allowable value can also be advantageously achieved, whereby the control behavior can be improved.

The controller or the control unit of the ventilator is particularly preferably configured to determine the second difference.

Preferably, the ventilator also has an expiratory line, wherein the first sensor is arranged or can be arranged in the expiratory line.

In this way, the inspiratory pressure can be determined particularly advantageously close to the patient. In particular when using the ventilator according to the invention in a therapy that only requires an inspiratory gas flow, the pressure in the expiratory line corresponds to the pressure at the patient or at the patient interface and thus advantageously allows the accuracy of the provision of information and thus the control to be increased.

The expiratory line can be a line of a two tube system as described above. Other lines as part of the tubing system are possible.

The ventilator may have further components, for example an inspiratory valve and/or an expiratory valve, additionally or alternatively a mixing chamber for mixing gases to obtain the respiratory gas and additionally or alternatively one or more fluid interfaces (flow interfaces) for supplying one or more gases to obtain the respiratory gas.

According to the invention, a process for controlling a ventilator for ventilating a patient through an inspiratory line is further provided. All advantages, effects, definitions, optional features and variants described in relation to the ventilator apply analogously to the process.

The process comprises the steps of providing a pressure signal indicating a pressure in the inspiratory line, providing a volume flow signal indicating a volume flow in the inspiratory line, determining a flow resistance of the inspiratory line based on the pressure signal and on the volume flow signal, receiving a first information indicating a predetermined target volume flow, receiving second information indicating a predetermined maximum allowable inspiratory pressure, determining an expected pressure based on the flow resistance and on the first and/or second information, and determining whether the expected pressure is higher than the maximum allowable inspiratory pressure; and/or determining a volume flow likely to occur based on the flow resistance and on the first and/or second information and determining a maximum allowable volume flow corresponding to the maximum allowable inspiratory pressure, and determining whether the volume flow likely to occur is higher than the maximum allowable volume flow; and providing a determination result corresponding to the determination. The process further comprises the step of controlling the pressure and/or the volume flow based on the determination result such that the maximum allowable inspiratory pressure and/or the maximum allowable volume flow is not exceeded.

Preferably, the ventilator, which is controlled by the process, is configured like the ventilator described above.

According to the invention, a system for ventilating a patient is further provided. The system comprises a ventilator as described above, a tubing system providing the inspiratory line and a patient interface.

All advantages, effects, definitions, optional features and variants described in relation to the ventilator apply analogously to the system.

In the context of the invention, a patient interface is understood to be a device which is configured to be mechanically and/or fluidically connected to the inspiratory line and at least part of a patient's head in order to guide the gas conducted through the inspiratory line to the patient. A patient interface can, for example, be configured as a breathing mask, a nasal mask or a nasal cannula.

The patient interface can be substantially sealing or non-sealing on or against the patient's face.

According to the invention, a computer program product is further provided. The computer program product comprises instructions that cause the above-described ventilator to perform the process steps of the above-described process. The computer program product may include a computer readable storage medium (or media) having non-transitory computer readable program instructions thereon for causing a processor (or one or more processors) to carry out aspects of the present invention. The computer readable storage medium can be a tangible device that can retain and store instructions for use by an instruction execution device such as the control unit or controller.

All advantages, effects, definitions, optional features and variants described in relation to the ventilator and the process apply analogously to the computer program product.

According to the invention, a computer-readable medium is also provided. The computer-readable medium stores or is a part of the computer program product described above.

All advantages, effects, definitions, optional features and variants described in relation to the ventilator and the process apply analogously to the computer-readable medium.

Further disclosed herein is a ventilator for ventilating a patient through an inspiratory line, the ventilator comprising a controller adapted to regulate the pressure and/or the flow rate in the inspiratory line such that a maximum allowable pressure in the inspiratory line and/or in an airway of the patient is not exceeded.

These and other features and effects can also be seen in the following description of the figures. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view of an embodiment of a ventilator according to the invention;

FIG. 2 is a schematic view of a further embodiment of a ventilator according to the invention;

FIG. 3 is a schematic view of a further embodiment of a ventilator according to the invention;

FIG. 4 is a schematic flow chart of an embodiment of a process according to the invention;

FIG. 5 is a schematic graph view in which an exemplary volume flow curve (volume flow course) over time is shown using a ventilator according to the invention; and

FIG. 6 is a schematic view of a further embodiment of a ventilator according to the invention.

### DESCRIPTION OF PREFERRED EMBODIMENTS

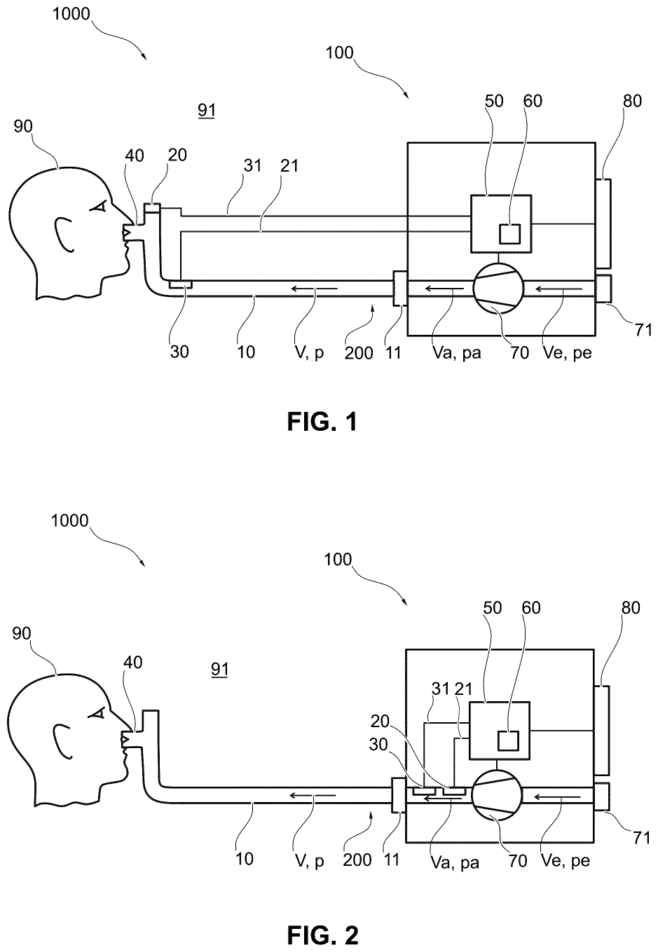

Referring to the drawings, according to the invention, a ventilator 100 is provided. Embodiments of such a ventilator 100 are shown in FIGS. 1 to 3 and 6.

The ventilator 100 according to the invention is used in each embodiment to ventilate a patient 90 through an inspiratory line 10. For this purpose, the inspiratory line 10 and the patient 90 may be fluidically and/or mechanically connected via a patient interface 40. The patient interface 40 can rest on or abut against at least a part of the head of the patient 90 in an essentially sealing manner with respect to the environment 91, or it can rest on or abut against at least a part of the head of the patient 90 in a non-sealing manner, as is the case, for example, with nasal prongs or a nasal cannulae.

The ventilator 100 has a breathing gas source 70. In the embodiments according to FIGS. 1, 2 and 3, the breathing gas source 70 can be a blower 70, which can be connected via at least one third fluid interface 71 to one or more gas sources, such as the environment 91 of the ventilator 100 and/or to one or more pressurized gas cylinders and/or to one or more supply lines of a hospital.

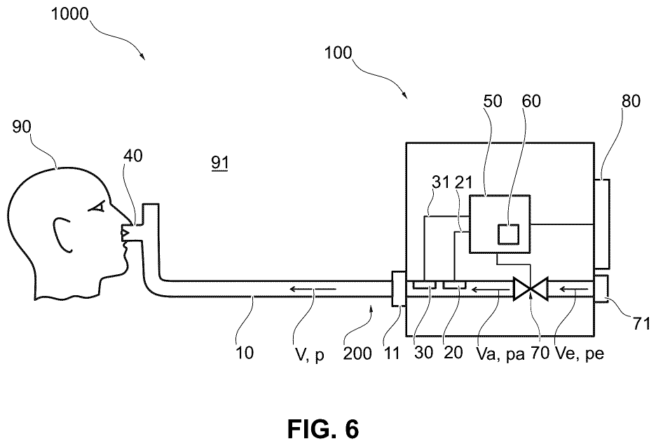

In the embodiment according to FIG. 6, the breathing gas source 70 may alternatively be a valve 70 which can be connected via at least one third fluid interface 71 to one or more gas sources, such as the environment 91 of the ventilator 100 and/or to one or more pressurized gas cylinders and/or to one or more supply lines of a hospital.

In each embodiment of a ventilator 100, a mixing chamber for mixing multiple gases may be provided between the at least one third fluid interface 71 and the breathing gas source 70.

In each embodiment of a ventilator 100, the breathing gas source 70 is thus configured to provide a gas or gas mixture intended for inspiration, namely the breathing gas, with suitable parameters on the device side, by means of which a pressure pe and/or volume flow Ve provided upstream of the breathing gas source 70 is controlled or regulated to a desired pressure pa and/or volume flow Va by the action of a controller (regulator) 60, which is yet to be described, on the breathing gas source 70. Thus, a gas flow flowing through the inspiratory line 10 can be provided with a pressure p in the inspiratory line 10 and with a volume flow V in the inspiratory line 10.

The ventilator 100 may further comprise a control unit 50, which may be configured to control and/or regulate components of the ventilator 100 for providing and/or influencing an inspiratory gas flow.

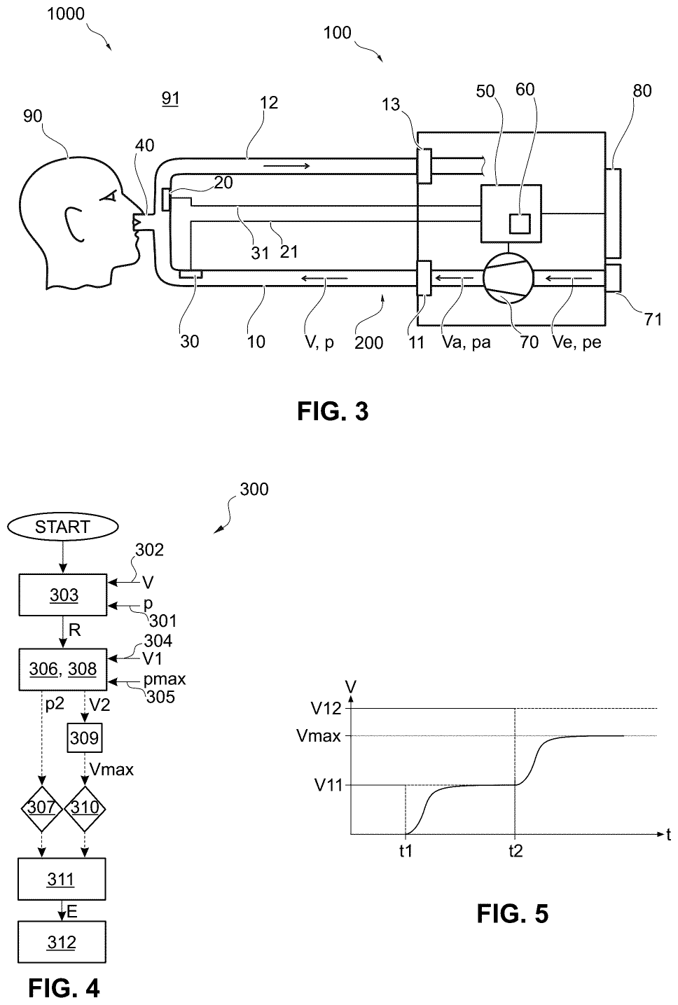

The ventilator 100 may comprise the inspiratory line 10 as part of a tubing system 200, as shown in the embodiments according to FIGS. 1, 2 and 6, or may further comprise additional lines, such as an expiratory line 12, as shown in the embodiment according to FIG. 3.

In each embodiment, the ventilator 100 can have fluid interfaces 11, 13 for receiving the inspiratory line 10 and/or the tubing system 200. In all embodiments, the respective ventilator 100 has, for example, a fluid interface 11 for connecting the inspiratory line 10, with the ventilator 100 according to FIG. 3 additionally having a second fluid interface 13 for connecting the expiratory line 12.

The fluid interface 11 may comprise an inspiratory valve or be provided by an inspiratory valve. It can be seen that the breathing gas source 70 in the sense of the invention is different from the inspiratory valve in that the breathing gas source 70 is provided upstream of the inspiratory valve.

In each embodiment, the ventilator 100 according to the invention has a first sensor 20 which is configured to provide a pressure signal which indicates a pressure p in the inspiratory line 10. The first sensor 20 can be an integral part of the ventilator 100, as shown in the embodiments according to FIGS. 2 and 6. However, the first sensor 20 can also be formed separately from the ventilator 100, as is the case in the embodiments according to FIGS. 1 and 3. In this case, the first sensor 20 is arranged, for example, in the inspiratory line 10 or in the expiratory line 12 or in the patient interface 40.

In each embodiment, the ventilator 100 according to the invention further comprises a second sensor 30 which is configured to provide a volume flow signal indicating a volume flow V in the inspiratory line 10. The second sensor 30 can be an integral part of the ventilator 100, as shown in the embodiments according to FIGS. 2 and 6. However, the second sensor 30 can also be formed separately from the ventilator 100, as is the case in the embodiments according to FIGS. 1 and 3. In this case, the second sensor 30 is arranged, for example, in the inspiratory line 10 or in the expiratory line 12 or in the patient interface 40.

The first sensor 20 and the second sensor 30 are connected to the ventilator 100, for example to the control unit 50 of the ventilator, via a data interface 21 and via a second data interface 31. Each of the data interfaces 21, 31 can be a wired or wireless interface.

In each embodiment, the ventilator 100 according to the invention further comprises a controller 60. It is particularly preferred and shown that the controller 60 is configured as a hardware module or software module of the control unit 50.

The controller 60 is configured to regulate the pressure p and/or the volume flow V based on the determination result E in such a way that the maximum allowable inspiratory pressure pmax and/or the maximum allowable volume flow Vmax is not exceeded.

In each embodiment, the ventilator 100 according to the invention is further configured to determine a flow resistance R of the inspiratory line 10 based on the pressure signal and on the volume flow signal, to receive first information indicating a predetermined target volume flow V1 and to receive second information indicating a predetermined maximum allowable inspiratory pressure pmax, and to determine a pressure p2 which is likely to occur based on the flow resistance R and on the first and/or second information and to determine whether the pressure p2 which is likely to occur is higher than the maximum allowable inspiratory pressure pmax and/or to determine a volume flow V2 which is likely to occur based on the flow resistance R and on the first and/or second information, whether the expected pressure p2 is higher than the maximum allowable inspiratory pressure pmax, and/or to determine an expected volume flow V2 based on the flow resistance R and on the first and/or second information and to determine whether the expected volume flow V2 is higher than a maximum allowable volume flow Vmax corresponding to the maximum allowable inspiratory pressure pmax and to provide a determination result E corresponding to the determination.

In each embodiment, the ventilator 100 according to the invention may further comprise an output unit 80, as shown in FIGS. 1 to 3. The output unit 80 can be configured to output the determination result E optically and/or acoustically. The output unit 80 can, for example, be configured as a display.

In this respect, FIG. 5 shows an example of the behavior of the ventilator 100 that can be achieved by the controller 60.

The flow rate V provided by the ventilator 100 in the inspiratory line 10 is shown. Up to time t1, the target volume flow, e.g. predetermined by a user, is zero in this example, so that the ventilator 100 does not provide any volume flow V during this period. At time t1, a target volume flow V11 other than zero is specified, which is lower than the maximum allowable volume flow Vmax. The controller 60 therefore regulates the gas flow by acting on the breathing gas source 70 so that the volume flow V provided in the regulated state is V11. In this example, at a later time t2, a higher target flow rate V12 is specified, which is higher than the maximum allowable flow rate Vmax. The controller 60 therefore regulates the gas flow in such a way that the volume flow V provided does not exceed the maximum allowable volume flow Vmax at any time. In the example shown, the control behavior is configured in such a way that the volume flow V approaches the target value (=the maximum allowable volume flow Vmax) degressively.

For example, in all embodiments, the ventilator 100 can be configured to determine a first difference between the predetermined target volume flow V1 and the maximum allowable volume flow Vmax, and to provide the first difference as a first input variable for the controller 60. In the example according to FIG. 5, the ventilator 100 can thus form a first difference between the setpoint volume flow V12 and the maximum allowable volume flow Vmax and provide this first difference as an input variable, for example as a disturbance variable, for the controller 60 so that the latter regulates the volume flow V in such a way that the maximum allowable volume flow Vmax is not exceeded at any time.

For example, in all embodiments, the ventilator 100 can be configured to determine a second difference between the maximum allowable inspiratory pressure pmax and the pressure p in the inspiratory line 10, and to provide the second difference as a second input variable for the controller 60. In this way, the degressive approximation of the volume flow V to the maximum allowable volume flow V3 shown as an example in FIG. 5 can be achieved in a particularly advantageous manner.

According to the invention, there is further provided a process 300 corresponding to the ventilator 100 for controlling a ventilator, in particular the ventilator 100 described above, for ventilating a patient 90 through an inspiratory line 10. An exemplary flowchart of a process 300 according to the invention is shown in FIG. 4.

Accordingly, the process 100 comprises the steps:
301 Providing a pressure signal which indicates a pressure p in the inspiratory line 10,
302 Providing a volume flow signal which indicates a volume flow V in the inspiratory line 10,
303 Determining a flow resistance R of the inspiratory line 10 based on the pressure signal and the volume flow signal,
304 Receiving a first information indicating a predetermined target volume flow V1, and
305 Receiving a second information indicating a predetermined maximum allowable inspiratory pressure pmax.

The process 300 further comprises the steps of: 306 determining an expected developing pressure p2 based on the flow resistance R and on the first and/or second information; and 307 determining whether the expected developing pressure p2 is higher than the maximum allowable inspiratory pressure pmax.

In addition to or as an alternative to steps 306, 307, the process 300 comprises the steps of: 308 determining an expected volume flow V2 based on the flow resistance R and on the first and/or second information, 309 determining a maximum allowable volume flow Vmax corresponding to the maximum allowable inspiratory pressure pmax, and 310 determining whether the expected volume flow V2 is higher than the maximum allowable volume flow Vmax.

The process 300 further comprises step: 311 providing a determination result E corresponding to the determination.

Optionally, the process 300 comprises the step of: outputting the determination result E, for example by means of the output unit 80.

The process 300 further comprises the step of: 312 controlling the pressure p and/or the volume flow V based on the determination result E such that the maximum allowable inspiratory pressure pmax and/or the maximum allowable volume flow Vmax is not exceeded.

The process 300 may further comprise the steps of: determining a first difference between the predetermined target volume flow V1 and the maximum allowable volume flow Vmax, and providing the first difference as a first input variable to the controller 60.

The process 300 may further comprise the steps of: determining a second difference between the maximum allowable inspiratory pressure pmax and the pressure p in the inspiratory line 10, and providing the second difference as a second input to the controller 60.

All of the features disclosed herein can be combined with each other as desired, provided that this does not affect alternatives or is contradictory.

While specific embodiments of the invention have been shown and described in detail to illustrate the application of the principles of the invention, it will be understood that the invention may be embodied otherwise without departing from such principles.

### LIST OF REFERENCE CHARACTERS

10 Inspiratory line
11 Fluid interface, inspiratory valve
12 Expiratory line
13 Second fluid interface
20 First sensor
21 First data interface
30 Second sensor
31 Second data interface
40 Patient interface
50 Control unit
60 Controller
70 Breathing gas source, blower, valve
71 Third fluid interface
80 Output unit
90 Patient
91 Environment
100 Ventilator
200 Tubing system
300 Procedure
301, 302, . . . . Process steps
1000 System
E Determination result
P Pressure in the inspiratory line
pe Pressure in the ventilator upstream of the breathing gas source
pa Pressure in the ventilator after (downstream of) the breathing gas source
pmax (Predetermined) Maximum allowable inspiratory pressure
p2 Expected pressure
t Time
t1, t2 Times
V Volume flow in the inspiratory line
Ve Volume flow in the ventilator upstream of the breathing gas source
Va Volume flow in the ventilator after the breathing gas source
V1, V11, V12 (Predetermined) Target volume flow
V2 Expected volume flow
Vmax Maximum allowable volume flow

What is claimed is:

1. A ventilator for ventilating a patient through an inspiratory line, the ventilator comprising:
a breathing gas source for supplying breathing gas;
a controller which acts as an actuator on the breathing gas source;
a first sensor which is configured to provide a pressure signal which indicates a pressure in the inspiratory line; and
a second sensor which is configured to provide a volume flow signal which indicates a volume flow in the inspiratory line,
wherein the ventilator is configured:
to determine a flow resistance of the inspiratory line based on the pressure signal and the volume flow signal;
to receive first information indicating a predetermined target volume flow;
to receive second information indicating a predetermined maximum allowable inspiratory pressure,
to determine, based on the flow resistance and based on the first information and/or the second information, an expected pressure and to determine whether the expected pressure is higher than the maximum allowable inspiratory pressure, and/or to determine, based on the flow resistance and based on the first information and/or the second information, an expected volume flow that is likely to occur and to determine whether the volume flow that is likely to occur is higher than a maximum allowable volume flow corresponding to the maximum allowable inspiratory pressure; and
to provide a determination result corresponding to the determination,
wherein the controller is configured to regulate the pressure and/or the volume flow based on the determination result such that the maximum allowable inspiratory pressure and/or the maximum allowable volume flow is not exceeded.

2. A ventilator according to claim 1, wherein the breathing gas source is configured as a blower or as a valve.

3. A ventilator according to claim 1, wherein the ventilator is configured:
to determine a first difference between the predetermined target volume flow and the maximum allowable volume flow; and
to provide the first difference as a first input variable for the controller.

4. A ventilator according to claim 3, wherein the ventilator is configured:
to determine a second difference between the maximum allowable inspiratory pressure and the pressure in the inspiratory line; and
to provide the second difference as a second input variable for the controller.

5. A ventilator according to claim 1, wherein the ventilator is configured:
to determine a difference between the maximum allowable inspiratory pressure and the pressure in the inspiratory line; and
to provide the difference as an input variable for the controller.

6. A ventilator according to claim 1, further comprising an expiratory line, wherein the first sensor is arranged in the expiratory line or is configured to be arranged in the expiratory line.

7. A ventilator according to claim 1, further comprising a computer program product comprising a computer readable storage medium (or media) having non-transitory computer readable program instructions thereon that cause the ventilator to:
determine a flow resistance of the inspiratory line based on the pressure signal and based on the volume flow signal;
receive the first information which indicates the predetermined target volume flow;
receive the second information indicating the predetermined maximum allowable inspiratory pressure;
determine the expected pressure based on the flow resistance and based on the first information and/or the second information, and determine whether the expected pressure is higher than the maximum allowable inspiratory pressure, and/or determine the volume flow expected to occur based on the flow resistance and based on the first information and/or the second information, and determine the maximum allowable volume flow corresponding to the maximum allowable inspiratory pressure, and determine whether the volume flow expected to occur is higher than the maximum allowable volume flow;
provide the determination result corresponding to the determination; and
regulate the pressure and/or the volume flow based on the determination result such that the maximum allowable inspiratory pressure and/or the maximum allowable volume flow is not exceeded.

8. A ventilator according to claim 1, in combination with a patient interface and a tubing system comprising the inspiratory line, wherein the combination forms a system for ventilating a patient.

9. A process for controlling a ventilator for ventilating a patient through an inspiratory line, the process comprising the steps of:
providing a pressure signal which indicates a pressure in the inspiratory line;
providing a volume flow signal which indicates a volume flow in the inspiratory line;
determining a flow resistance of the inspiratory line based on the pressure signal and based on the volume flow signal;
receiving first information which indicates a predetermined target volume flow;
receiving second information indicating a predetermined maximum allowable inspiratory pressure;
determining an expected pressure based on the flow resistance and based on the first information and/or the second information, and determining whether the expected pressure is higher than the maximum allowable inspiratory pressure, and/or determining a volume flow expected to occur based on the flow resistance and based on the first information and/or the second information, and determining a maximum allowable volume flow corresponding to the maximum allowable inspiratory pressure, and determining whether the volume flow expected to occur is higher than the maximum allowable volume flow;
providing a determination result corresponding to the determination; and
regulating the pressure and/or the volume flow based on the determination result such that the maximum allowable inspiratory pressure and/or the maximum allowable volume flow is not exceeded.

10. A process according to claim 9, wherein the ventilator comprises a breathing gas source for supplying breathing gas, a controller which acts as an actuator on the breathing gas source; a first sensor configured to provide the pressure signal which indicates the pressure in the inspiratory line; and a second sensor which is configured to provide the volume flow signal which indicates the volume flow in the inspiratory line,
wherein the ventilator is configured:
to determine the flow resistance of the inspiratory line based on the pressure signal and the volume flow signal;
to receive the first information indicating the predetermined target volume flow;
to receive the second information indicating the predetermined maximum allowable inspiratory pressure,
to determine, based on the flow resistance and based on the first information and/or the second information, the expected pressure and to determine whether the expected pressure is higher than the maximum allowable inspiratory pressure, and/or to determine, based on the flow resistance and based on the first information and/or the second information, the expected volume flow that is likely to occur and to determine whether the volume flow that is likely to occur is higher than a maximum allowable volume flow corresponding to the maximum allowable inspiratory pressure; and
to provide a determination result corresponding to the determination,
wherein the controller is configured to regulate the pressure and/or the volume flow based on the determination result such that the maximum allowable inspiratory pressure and/or the maximum allowable volume flow is not exceeded.

11. A process according to claim 9, wherein the breathing gas source is configured as a blower or as a valve.

12. A process according to claim 9, wherein the ventilator is configured:
to determine a first difference between the predetermined target volume flow and the maximum allowable volume flow; and
to provide the first difference as a first input variable for the controller.

13. A process according to claim 12, wherein the ventilator is configured:
to determine a second difference between the maximum allowable inspiratory pressure and the pressure in the inspiratory line; and
to provide the second difference as a second input variable for the controller.

14. A process according to claim 9, wherein the ventilator is configured:
to determine a difference between the maximum allowable inspiratory pressure and the pressure in the inspiratory line; and
to provide the difference as an input variable for the controller.

15. A process according to claim 9, further comprising an expiratory line, wherein the first sensor is arranged in the expiratory line or is configured to be arranged in the expiratory line.

16. A process according to claim 9, wherein the ventilator is in combination with a patient interface and a tubing system comprising the inspiratory line and the combination forms a system for ventilating a patient.

17. A process according to claim 9, further comprising providing a computer program product comprising a computer readable storage medium (or media) having non-transitory computer readable program instructions thereon that cause the ventilator to perform at least some of the process steps.

18. A computer program product comprising a computer readable storage medium (or media) having non-transitory computer readable program instructions thereon that cause a ventilator to:
provide a pressure signal which indicates a pressure in an inspiratory line;
provide a volume flow signal which indicates a volume flow in the inspiratory line;
determine a flow resistance of the inspiratory line based on the pressure signal and based on the volume flow signal;
receive first information which indicates a predetermined target volume flow;
receive second information indicating a predetermined maximum allowable inspiratory pressure;
determine an expected pressure based on the flow resistance and based on the first information and/or the second information, and determine whether the expected pressure is higher than the maximum allowable inspiratory pressure, and/or determine a volume flow expected to occur based on the flow resistance and based on the first information and/or the second information, and determine a maximum allowable volume flow corresponding to the maximum allowable inspiratory pressure, and determine whether the volume flow expected to occur is higher than the maximum allowable volume flow;
provide a determination result corresponding to the determination; and
regulate the pressure and/or the volume flow based on the determination result such that the maximum allowable inspiratory pressure and/or the maximum allowable volume flow is not exceeded,
wherein the ventilator comprises a breathing gas source for supplying breathing gas, a controller which acts as an actuator on the breathing gas source; a first sensor configured to provide the pressure signal which indicates the pressure in the inspiratory line; and a second sensor which is configured to provide the volume flow signal which indicates the volume flow in the inspiratory line,
wherein the ventilator is configured:
to determine the flow resistance of the inspiratory line based on the pressure signal and the volume flow signal;
to receive the first information indicating the predetermined target volume flow;
to receive the second information indicating the predetermined maximum allowable inspiratory pressure,
to determine, based on the flow resistance and based on the first information and/or the second information, the expected pressure and to determine whether the expected pressure is higher than the maximum allowable inspiratory pressure, and/or to determine, based on the flow resistance and based on the first information and/or the second information, the expected volume flow that is likely to occur and to determine whether the volume flow that is likely to occur is higher than the maximum allowable volume flow corresponding to the maximum allowable inspiratory pressure; and
to provide the determination result corresponding to the determination,
wherein the controller is configured to regulate the pressure and/or the volume flow based on the determination result such that the maximum allowable inspiratory pressure and/or the maximum allowable volume flow is not exceeded.
